(19) United States
(12) Patent Application Publication
(10) Pub. No.: US 2004/0234592 A1
(43) Pub. Date: Nov. 25, 2004

(54) Dermatological formulations

(76) Inventor: Bo Carlsson, Stockholm (SE)

Correspondence Address: WIGGIN AND DANA LLP, ATTENTION: PATENT DOCKETING, ONE CENTURY TOWER, P.O. BOX 1832, NEW HAVEN, CT, 06508-1832, US

(21) Appl. No.: 10/475,961

(22) PCT Filed: May 3, 2002
(86) PCT No.: PCT/EP02/04954

(30) Foreign Application Priority Data
May 9, 2001 (GB) 0111314.1

(51) Int. Cl. A61K 31/198; A61K 9/20

(52) U.S. Cl. 424/465; 514/567

(57) ABSTRACT

A composition comprising at least one thyroid hormone compound or thyroid hormone-like compound, a hydrophilic phase-forming component, an amino alcohol and at least two emulsifying or emollient excipients selected from the group consisting of mineral oil, C₁₂-C₂₄ alcohols, C₁₂-C₂₄ carboxylic acids, C₁-C₈ branched or linear alkyl esters of C₁₂-C₂₄ carboxylic acids, glyceryl esters of C₁₂-C₂₄ carboxylic acids, macrogel ethers, polyethylene glycol esters of C₁₂-C₂₄ carboxylic acids, sorbitan esters of C₁₂-C₂₄ carboxylic acids (Span compounds) and polyoxyethylenated sorbitan esters of C₁₂-C₂₄ carboxylic acids (Tween compounds). A preferred thyroid hormone or thyroid hormone-like compound is triiodothyroacetic acid (TriAc; 4-(4-hydroxy-3-iodophenoxy-3,5-diiodophenyl)acetic acid) and a preferred amino alcohol is triethanolamine.

### TECHNICAL FIELD

The present invention relates to skin care preparations. In particular, it concerns topical formulations containing thyroid hormone and thyroid hormone-like compounds for the treatment of skin disorders, as a preventive treatment prior to cosmetic surgery, as a concomitant treatment with topical corticosteroids and for cosmetic use. A preferred preparation comprises Triiodothyroacetic acid (TriAc) as active ingredient.

### BACKGROUND

In WO 96/40048, thyroid hormone and thyroid hormone-like compounds, particularly the thyroid hormone analogue triiodothyroacetic acid ([4-(4-Hydroxy-3-iodophenoxy)-3,5-diiodophenyl]acetic acid with CAS number 51-24-1 and hereafter referred to as TriAc) are shown to have biological activity in skin when applied in topical formulations. In established models of human skin, TriAc was found to regulate expression of several genes that are important for skin structure and function. Other thyroid hormone compounds and thyroid hormone-like compounds were shown to have similar effects to TriAc in various in vitro and in vivo tests.

There are a considerable number of potential uses for dermatologically active thyroid hormone compounds and thyroid hormone-like compounds. These include treatment of corticosteroid induced skin atrophy, actinic skin damage, intrinsically aged skin, wrinkled skin, collagen deficient skin, stria, cellulite, roughened skin and skin scarring. The compounds may be able to heal skin bruising and micro tears and increase skin thickness, preventing the development of skin ulcers in diabetics with diabetic dermopathy. They may also be used as pretreatment prior to planned cosmetic surgery, such as CO₂ laser resurfacing, and may also be used as a concomitant treatment with topical corticosteroids for diseases such as psoriasis and eczema to reduce the atrophying effects. This latter effect will make it possible to extend the treatment period with the corticosteroid and to broaden the otherwise restricted use of potent topical corticosteroids on particular areas of the body, such as the face, and in patients with sensitive skin, notably children.

In a set of experiments in mice it has been shown that concomitant treatment with TriAc totally prevents the atrophy (manifest as a reduction of total collagen and of cross-linking between the collagen bundles in the dermis) induced by treatment with topical betamethasone (a potent corticosteroid).

In order to realise the potential dermatological benefits of these compounds, it is necessary to present them in compositions with acceptable stability, toxicological, rheological, cosmetic and drug delivery characteristics. Such compositions are not provided by the prior art.

In U.S. Pat. No. 5,883,294, a topical composition containing a thyroid hormone agonist is disclosed. The composition comprises an oil phase, surfactants and water. U.S. Pat. No. 5,322,689 discloses topical compositions for the release of aromatic compounds for the treatment of respiratory disorders. One composition contains triethanolamine in combination with water and two fatty alcohols but is not suggested to be of any utility for the delivery of compounds other than those useful in respiratory disease.

It is therefore an object of the present invention to provide compositions having such characteristics.

In accordance with one aspect of the invention, there is provided a composition comprising at least one thyroid hormone compound or thyroid hormone-like compound, a hydrophilic phase-forming component, an amino alcohol and at least two emulsifying or emollient excipients selected from the group consisting of mineral oil, C₁₂-C₂₄ alcohols, C₁₂-C₂₄ carboxylic acids, C₁-C₈ branched or linear alkyl esters of C₁₂-C₂₄ carboxylic acids, glyceryl esters of C₁₂-C₂₄ carboxylic acids, macrogol ethers, polyethylene glycol esters Qf C₁₂-C₂₄ carboxylic acids, sorbitan esters of C₁₂-C₂₄ carboxylic acids (Span compounds) and polyoxyethylenated sorbitan esters of C₁₂-C₂₄ carboxylic acids (Tween compounds).

As used herein, the term ‘thyroid hormone compound or thyroid hormone-like compound’ refers to a chemical entity which binds to the TRα receptor or the TRβ receptor with a dissociation constant, K_{d}, lower than 1 μM, wherein

K_{d}=(R)·(L)/(RL),

where (R) is the concentration of receptor, (L) is the concentration of ligand, and (RL) is the concentration of the receptor-ligand complex.

The amino alcohol of the composition of the present invention preferably has from 2 to 6 carbon atoms in each alcohol chain. The hydroxyl group of the amino alcohol is preferably situated on the carbon atom furthest from the amino nitrogen. The amino alcohol may be an ethanolamine and is preferably triethanolamine.

The hydrophilic phase-forming component preferably comprises water. Preferred emulsifying or emollient excipients include octyl palmitate, isopropyl myristate, isopropyl palmitate, cetostearyl alcohol, Cetearth-20, Cetromacrogol 1000, glyceryl monostearate, cetyl alcohol, stearic acid, glyceryl stearate and PEG-100 stearate.

Preferably, the amino alcohol, such as triethanolamine, is present in the composition at a level of 0.1 to 5% w/W. The composition may also comprise a humectant, such as sorbitol, which will preferably be present at a concentration of 1 to 5% w/w. A thickening agent may also be present and may comprise a synthetic polymer such as a Carbomer or Carbopol. The thickening agent will preferably be present at a concentration of 0.1 to 1% w/w. Preservative ingredients may also be included and these may be selected from methylparaben, propylparaben, imidurea and 1-(3-chloroallyl)-3,5,7-triaza-1-azoniaadomaniane chloride (CAS# 51229-78-8). The preservative ingredients will preferably be incorporated into the composition at a level of 0.1 to 0.5% w/w.

The composition may also include EDTA, preferably at a level of 0.02 to 2% w/w. The inclusion of one or more antioxidants is also preferred. The antioxidants may be selected from butylated hydroxyanisole, butylated hydroxytoluene, n-propyl trihydroxybenzoate, t-butylhydroquinone (all of which have preferred concentrations of 0.01 to 0.05% w/w) and d1-α,δ,γ-tocopherol (which has a preferred concentration of 0.1 to 2% w/w).

A UV-filter component may be incorporated into the composition. The UV-filter component may comprise one or more compounds selected from aminobenzoic acid, dioxybenzone, oxybenzone, sulisobenzone, diethanolamine methoxycinnamate, ethyldihydroxypropyl aminobenzoate, glyceryl aminobenzoate, octyl dimethyl aminobenzoate, trolamine salicylate and octyl methoxycinnamate, all of which have preferred concentrations of 0.01 to 1% w/w.

The at least one thyroid hormone compound or thyroid hormone-like compound is preferably selected from Tri-iodothyronine (3,5,3′-triiodothyronine) (T3); D and L tetraiodothyronine-thyroxine (T4); 3, 3′, 5′ tri-iodothyronine (reverse T3); 3, 3′-diiodothyronine; T3 T4 analogues such as 3, 5, 3′-Triiodothyroacetic acid; 3, 5, 3′-Tetraiodothyroacetic acid; 3, 5, 3′-Triiodo-L-thyronine; 3, 5, 3′-Triiodo-L-thyronine methyl ester; 3, 5, 3′-Triiodo-L-thyronine hydrochloride; L-thyroxine; L-thyroxine hydrochloride; L-T3; T4Fo; Tetrac (3-[4-(4-hydroxy-3,5-diiodophenoxy)-3, 5-diiodophenyl]acetic acid); Triac ([4-(4-hydroxy-3-iodophenoxy)-3,5-diiodophenyl]acetic acid); Tetraprop; Triprop ([4-(4-hydroxy-3-iodophenoxy)-3,5-diiodophenyl]propionic acid); T4Bu; T3Bu; Thyroxamine; Triiodothyronamine; L-3′-T1′; L-3′-T1; L-3,5′-T2; L-3,3′-T2; L-3,3′,5′-T3; DL-Br2I; L-Br2iPr; L-Me2I; L-Me3; L-Me4; L-Me2IPr; DL-IMEI; L-3,5-Dimethyl-3′-isopropylthyronine (DIMIT); DL-BPT4; B-triac; BP-tetrac; DL-SBT3; DL-SBT4, DL-MBT3, MB-tetrac; T2; T2F; T2CI; T2Br; T3; T2Me; T2Et; T21Pr; T2nPr; T2sBu; T2tBu; T21Bu; T2Phe; T2OH; T2NO2; T2F2; T2CI2; T4; T2Me2; 3, 5, 3′,5′-tetraiodo-D-thyronine; 3,5,3′-Triiodo-D-thyronine; 3,5-Diiodo-4-hydroxyphenylpropionic acid (DIHPA); Aryloxamic acids; (arylamino) acetic acids; arylpropionic acids; arylthioacetic acids; (aryloxy) acetic acid; 3,3′-T2; 3,5-T2; 3′-5′-T2; (5-Benzyloxy-2-methoxyphenyl)-(2-methoxypyrimidin-5-yl)-methanol; Benzyloxy-2-methoxyphenyl)-(6-methylpyridin-3-yl)methanol; (5-Benzyloxy-2-methoxyphenyl)-(5-bromo-2-methoxypyridin-4-yl)methanol; (5-benzyloxy-2-methoxyphenyl)-2,6-difluoropyridin-3-yl)methanol; (5-Benzyloxy-2-methoxyphenyl)-(2-methoxypyridin4-yl) methanol; 4-Methoxy-3-[(2-methoxypyrimidin-5-yl)methyl]phenol; 4-Methoxy-3-[(6-methylpyrid-3-yl)methyl]phenol; 5-Benzyloxy-2-methoxybenzyl Bromide; (5-Benzyloxy-2-methozyphenyl)-(6-chloropyridazin-3-yl)-acetonitrile; 4-Benzyloxy-2-[2-methoxythiazol-5-yl)methyl]anisole; 6-[(5-Hydroxy-2-methoxyphenyl)methyl]thiazol-2-(31H); 3′-Heteroarylmethyl-4′-)-methyl-3,5-dinitro-N-trifluoro-acetyl-L-thyronine Ethyl Esters; 3′-heteroarylmethyl-3,5-di-iodo-4′)-methyl-N-trifluoro-acetyl-L-thyronine Ethyl Esters; 3′-heteroarylmethyl analogues of 3,3′,5-tri-iodo-L-thyronine (T3); 3′-substituted derivatives of the thyroid hormone 3,3′,5-triiodo-L-thyronine (T3); L-3′-TI; L-3,5′-T2; L-3,3′-T2; L-3,3′,5′-T3; DL-Br2I; L-Br2IPr; L-Me2I; L-Me3; L-Me4; L-Me2IPr; DL-IMeI; L-3,5-Dimethyl-3′-isopropylthyronine (DIMIT); DL-BPT4; B-triac; BP-tetrac; DL-SBT3; DL-SBT4; DL-MBT3; MB-tetrac; T2; T2F; T2CI; T2Br; T3; T2Me; T2Et; T2IPr; T2nPr; T2sBu; T2tBu; T2IBu; T2Phe; T2OH; T2NO2; T2F2; T2CI2; T4; T2Me2; 3,5,3′,5′-tetraiodo-D-thyronine; 3,5,3′-Triiodo-D-thyronine; 3,5-Diiodo-4-hydroxyphenylpropionic acid (DIHPA); Aryloxamic acids; (arylamino) acetic acids; arylpropionic acids; arylthioacetic acids; (aryloxy) acetic acid; 3,3′-T2; 3,5-T2; 3′-5′-T2; α-methyl-3,5,3′-triiodothyroacetic acid, α-methyl-3,5,3′-triiodothyropropionic acid, and α-methyl-3,5,3′5′-tetraiodothyropropionic acid; methylene- and carbonyl-bridged analogs of iodinated thyronines or thyroacetic acids; or iodinated benzofurans; 3,5-diiodo-4-(2-N,N-diethylaminoethoxy)phenyl-(2-butylbenzofur-3-yl)methanol hydrochloride; 2-methyl-3-(3,5-diiodo-4-(2-N,N-diethylaminoethoxy)-benzoyl) benzofuran; hydrochloride; 2-n-butyl-3-(3,5-diiodo-4-carboxymethoxy-benzoyl)benzofuran; 2-methyl-3-(3,5-diiodo-4-carboxymethoxy-benzyl)benzofuran; [4′-hydroxy-3′-iodo-3, 5 diido-4-(2-N,N-dimethylamino-(ethoxy)benzophenon hydrochloride; 2-butyl-3-(3-iodo-4-hydroxybenzoyl)benzofuran; 4′4-dihydroxy 3′3,5-triiodo-diphenylmethane; 3,5-diiodo-4-(2-N,N-diethylaminoethoxy)phenyl-; -(2-butylbenzofur-3-yl)methanol hydrochloride; 2-n-butyl-3-(3,5-diiodo-4-carboxymethoxy-benzoyl)benzofuran; 2-methyl-3-(3,5-diiodo-4-hydroxy-benzoyl)benzofuran; 2-methyl-3-(3,5-diiodo-4-carboxymethoxy-benzyl)benzofuran; 4′hydroxy-3′-iodo-3,5 diiodo-4-(2-N, N-dimethylamino-ethoxy)benzophenon hydrochloride; 2-butyl-3-(3-iodo-4-hydroxybenzoyl)benzofuran; 4′,4-dihydroxy-3′3,5-triiodo-diphenylmethan; 3,5-diethyl, 3′-isopropyl thyronine (DIET); and IpTA2 (3,5 diiodo-3′ isopropyl thyroacetic acid) and pharmacologically acceptable salts and derivatives thereof.

More preferably, one of the thyroid hormone or thyroid hormone-like compounds is triodothyroacetic acid (TriAc). The TriAc is preferably present at a concentration of 0.001 to 0.3% w/w, more preferably 0.01 to 0.3% w/w.

According to another aspect of the invention, there is provided a method of predicting the efficacy of topical compositions comprising thyroid hormone or thyroid hormone-like compounds, the method comprising contacting the compositions with a stack of dolichol/propylene glycol gel membranes, incubating the compositions and membranes to allow diffusion of the thyroid hormone or thyroid hormone-like compounds into the membranes, extracting the diffused thyroid hormone or thyroid hormone-like compounds from the membranes and measuring the amount of extracted diffused thyroid hormone or thyroid hormone-like compounds.

The efficacy-predicting method may be applied to different batches of the same composition to analyse for batch-to-batch variations in predicted efficacy. It may also be used to evaluate and compare different compositions containing the same thyroid hormone or thyroid hormone-like compounds. In this way, compositions prepared by different manufacturers may be compared.

The ideal topical formulation should have appealing cosmetic features, acceptable pharmaceutical features, be non-irritating and non-toxic and easy to produce in large quantities in accordance with Good Manufacturing Practice for medical topical formulations.

The composition of the present invention preferably comprises an oil-in-water cream-base into which the thyroid hormone or thyroid hormone-like compound (in different concentrations) is incorporated.

### EXAMPLES

The invention will now be described in more detail by way of example only and with reference to the appended drawings, of which:

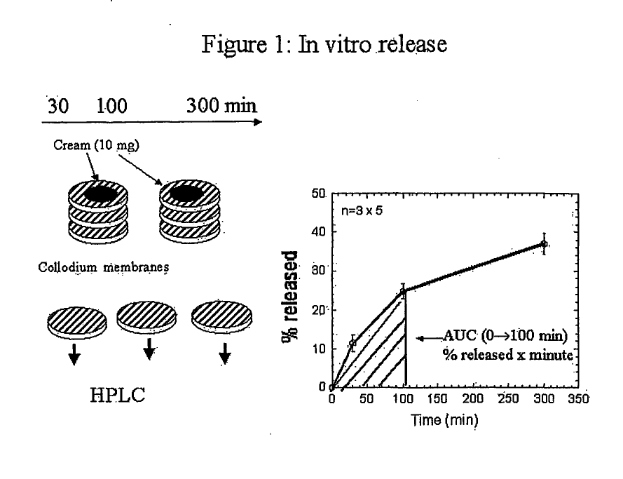

FIG. 1 shows a schematic representation of the efficacy-predicting method of the present invention, along with a sample of experimental results obtained thereby; and

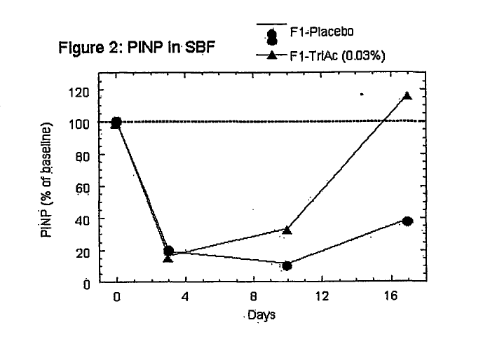

FIG. 2 shows the result of an in vivo study into the effect of a composition of the present invention on betamethasone-induced changes in collagen synthesis in dermal fibroblasts.

### Example 1

### Formulation Development

Introduction

The most important advantage of creams is their high degree of patient acceptance due to fine texture, pleasant feel and elegant appearance. Creams are semisolid emulsions and may be oil-in-water or water-in-oil. An oil-in-water formulation typically contains water, emulsifiers, emollients, surfactants and gelling-agents constituting the cream-base. Preservatives are often added to prevent deterioration due to microorganisms when the consumer uses the cream. Chelating agents can be added as scavengers for metal compounds that can cause coloration or precipitation. Anti-oxidants are frequently included in creams to prevent rancidity and to increase stability of sensitive ingredients. UV filters may be included in creams to prevent degradation of ingredients due to UV-radiation.

It is crucial that creams used as vehicles for topical delivery of an active ingredient (in this example, TriAc) allow the active ingredient to be released from the cream into the skin. The release-rate of an active ingredient from the vehicle into the skin is dependent in a complex way on numerous factors such as diffusion, solubility and partitioning. Creams composed of identical ingredients may display different release-profiles dependent on how they have been manufactured. Therefore, an assay (the multilayer membrane system (MMS)-model) was invented and used to evaluate TriAc-release from standard-formulations and the assay results were used as an iterative tool in formulation development.

Results

Eight oil-in-water cream formulations (see Table 1) were developed.

Manufacture of the formulations started with heating the water to 75-85° C. under stirring then adding methylparaben, propylparaben, EDTA, thickening agent (Carbopol) and sorbitol. The emollients and emulsifiers were then blended at 75-85° C. and this oil-phase was added to the water-phase. Then triethanolamine was added to adjust pH to around 6.8-7.1. The vessel temperature was then reduced to 60° C. and imidurea or Promulgen was added.

Two pilot-formulations (P1 and P2) were developed. TriAc was dissolved in isopropanol (70%) and incorporated in the creams. The release-rate of TriAc from the cream (see example 2) was considered as an important parameter when deciding which formulation should be selected for further development. Both creams displayed an excellent release of TriAc. Six variants (F1 to F6) of the two pilot-formulations were then manufactured and tested in the same way. It was found that F1 displayed the best release rate and this formulation was selected for future development.

F1 has been produced in four different concentrations of TriAc (0, 0.03%, 0.1% and 0.3%) for local tolerance tests (see example 4) and for clinical trials. Large scale batches (7 kg cream) at 0 (placebo), 0.03% and 0.1% TriAc have been produced on three different occasions. Essentially the same manufacturing protocol as described above has been used with addition of TriAc in isopropanol (or isopropanol alone for placebo-product) as the last step. An alternative way to add TriAc to the formulation is to add it to the oil-phase at 75-850 C. This is more convenient when manufacturing on an industrial scale and has been tested with excellent results (see example 2).

The efficacy of this formulation (TriAc in F1) in human skin has been evaluated in clinical trials (see example 5).

Data collected in stability studies of F1-TriAc (0, 0.03% and 0.1%) and of key ingredients (TriAc, propylparabens, methylparabens and imidurea) indicates that the products are stable for more than 12 months (see example 3).

TABLE 1
List of ingredients (as % w/w) used in the manufacture of the eight formulations of Example 1.

| Ingredient | | P1 | P2 | F1 | F2 | F3 | F4 | F5 | F6 |
|---|---|---|---|---|---|---|---|---|---|
| Deionized water | | 77-79 | 77-79 | 77-79 | 77-79 | 77-79 | 77-79 | 77-79 | 77-79 |
| Arlacel 165 | Emulsifier | | 3.50 | | 3.50 | | 3.50 | 3.50 | 3.50 |
| Octyl Palmitate | Emollient | 5.00 | 10.00 | 5.00 | 10.00 | | | 10.00 | |
| Isopropyl Myristate | Emollient | | | | | 5.00 | | | |
| Isopropyl Palmitate | Emollient | | | | | | 8.00 | | 8.00 |
| Mineral oil | Emollient | | | | | | 2.00 | | 2.00 |
| Promulgen D | Emulsifier/ | | 3.00 | | 3.00 | | | | 3.00 |
| | Emollient | | | | | | | | |
| Cetostearyl Alcohol | Emulsifier/ | | | 1.00 | | 1.00 | 2.00 | 2.00 | |
| | Emollient | | | | | | | | |
| Cetomacrogol 1000 | Emulsifier | | | | | | 1.00 | 1.00 | |
| Glyceryl | Emulsifier/ | 6.00 | | 6.00 | | 6.00 | | | |
| Monostearate | Emollient | | | | | | | | |
| Cetyl alcohol | Emulsifier/ | 3.00 | 1.00 | 2.00 | | 2.00 | | | |
| | Emollient | | | | | | | | |
| Stearic acid | Emulsifier | 3.00 | | 3.00 | | 3.00 | | | |
| Sorbitol | Humectant | 2.8 | 2.8 | 2.8 | 2.8 | 2.8 | 2.8 | 2.8 | 2.8 |
| Triethanolamine | Neutralising | 0.92 | 0.48 | 0.4-0.8 | 0.4 | 0.7 | 0.6 | 0.7 | 0.2 |
| | Base/ | | | | | | | | |
| | Surfactant | | | | | | | | |
| Methylparaben | Preservative | 0.20 | 0.20 | 0.20 | 0.20 | 0.20 | 0.20 | 0.20 | 0.20 |
| Propylparaben | Preservative | 0.10 | 0.10 | 0.10 | 0.10 | 0.10 | 0.10 | 0.10 | 0.10 |
| Dowicil 200 | Preservative | 0.2 | 0.2 | | | | | | |
| Imidurea | Preservative | | | 0.3 | 0.3 | 0.3 | 0.3 | 0.3 | 0.3 |
| Disodium EDTA | Chelating | 0.2 | 0.2 | 0.2 | 0.2 | 0.2 | 0.2 | 0.2 | 0.2 |
| | Agent | | | | | | | | |
| Isopropanol | Solvent | 0.7 | 0.7 | 0.7 | 0.7 | 0.7 | 0.7 | 0.7 | 0.7 |
| Carbopol 940 | Thickening | 0.2 | 0.3 | | | | | | |
| | Agent | | | | | | | | |
| Carbopol 980 | Thickening | | | 0.2 | 0.2 | 0.2 | 0.2 | 0.2 | 0.2 |
| | Agent | | | | | | | | |
| TriAc | Active | | | 0.03/0.1 | | | | | |
| | ingredient | | | | | | | | |
| pH | | pH = 7.0 | pH = 6.7 | pH = 6.1-7.1* | pH = 6.7 | pH = 6.7 | pH = 6.7 | pH = 6.8 | pH = 6.8 |

Note:
Arlacel 165 is a mixture of Glyceryl Stearate & PEG-100 Stearate
Dowicil 200 is a preservative (94%, 1-(3-chloroallyl)3,5,7-triaza-1-azoniaadomaniane chloride).
Promulgen D is a commercially produced mixture of cetostearyl alcohol (CAS 267/008/16) and Cetearth-20 (CAS 200/849/9, INCI name: ethoxylated (20 moles) cetyl/stearyl alcohol).
*Preferred pH-interval.

TABLE 2
Comparison between 11 different products for TriAc release in the MMS-model.

| | Strength | | Released | Released | Released | AUC 0→100 min | AUC 0→100 min |
|---|---|---|---|---|---|---|---|
| Product | (% TriAc) | TriAc added | 30 min | 100 min | 300 min | μg * min | % * min |
| Essex-cream | | 10 μg | 1.1 +/− 0.2 μg | 2.5 +/− 0.2 μg | 3.2 +/− 0.3 μg | 144 +/− 11 | |
| | 0.1% | | (11 +/− 2%) | (25 +/− 2%) | (32. +/− 3%) | | 1436 +/− 108 |
| TriAcana ™ | | 20 μg | 1.4 +/− 0.4 μg | 5.5 +/− 0.6 μg | 11 +/− 0.6 μg | 265 +/− 37 | |
| | 0.2% | | (8.1 +/− 2.5%) | (31 +/− 3.2%) | (63 +/− 3.4%) | | 1480 +/− 209 |
| F1 | | 10 μg | 3.5 +/− 0.5 μg | 5.5 +/− 0.4 μg | 5.5 +/− 0.2 μg | 371 +/− 43 | |
| pilot batch | 0.1% | | (35.4 +/− 5%) | (55.2 +/− 4%) | (54.9 +/− 2%) | | 3706 +/− 427 |
| F1A | | 10 μg | 3.9 +/− 0.4 μg | 6.5 +/− 0.4 μg | 6.9 +/− 0.3 μg | 425 +/− 19 | |
| | 0.1% | | (39.3 +/− 4%) | (65.3 +/− 5%) | (69.5 +/− 3%) | | 4255 +/− 187 |
| F1D | | 3.3 μg | 1.3 +/− 0.1 μg | 1.9 +/− 0.1 μg | 2.2 +/− 0.1 μg | 131 +/− 8 | |
| | 0.03% | | (43.1 +/− 4%) | (63.2 +/− 4%) | (72.8 +/− 2%) | | 4365 +/− 264 |
| F1B | | 10 μg | 4.6 +/− 0.3 μg | 6.7 +/− 0.4 μg | 6.6 +/− 0.3 μg | 463 +/− 30 | |
| | 0.1% | | (45.8 +/− 3%) | (66.9 +/− 4%) | (65.5 +/− 3%) | | 4633 +/− 295 |
| F1E | | 3.3 μg | 1.3 +/− 0.2 μg | 2.1 +/− 0.2 μg | 2.0 +/− 0.1 μg | 138 +/− 15 | |
| | 0.03% | | (44.0 +/− 6%) | (69.0 +/− 6%) | (66.2 +/− 4%) | | 4616 +/− 510 |
| F1C | | 10 μg | 5.1 +/− 0.2 μg | 7.1 +/− 0.4 μg | 7.2 +/− 0.2 μg | 503 +/− 24 | |
| | 0.1% | | (50.9 +/− 2%) | (71.0 +/− 4%) | (72.3 +/− 2%) | | 5028 +/− 268 |
| F1F | | 3.3 μg | 1.4 +/− 0.1 μg | 2.1 +/− 0.1 μg | 2.0 +/− 0.1 μg | 143 +/− 11 | |
| | 0.03% | | (46.1 +/− 5%) | (70.4 +/− 4%) | (66.9 +/− 3%) | | 4772 +/− 370 |
| F1TestA | | 10 μg | 4.1 +/− 0.4 μg | 7.3 +/− 0.5 μg | 7.3 +/− 0.1 μg | 461 +/− 10 | |
| | 0.1% | | (41.4 +/− 4%) | (72.7 +/− 5%) | (73.2 +/− 1%) | | 4614 +/− 101 |
| F1TestB | | 10 μg | 4.9 +/− 0.6 μg | 7.2 +/− 0.2 μg | 7.1 +/− 0.2 μg | 496 +/− 31 | |
| | 0.1% | | (48.9 +/− 6%) | (71.9 +/− 2%) | (71.0 +/− 2%) | | 4962 +/− 309 |

### Example 2

### In-Vitro Release Tests

Introduction

The ultimate objective for formulation development has been to develop a topical formulation of TriAc to be used to influence gene-expression in the skin. In order to achieve this it is necessary that the formulation allows the active ingredient (TriAc) to be delivered from the formulation to the target cells in the dermis and epidermis. The skin consists primarily of three different layers: the dermis (fibroblasts), the epidermis (keratinocytes) and the stratum corneum which consists of layers of dead keratinocytes. The stratum corneum constitutes the body's barrier towards the environment and a topically applied drug must penetrate this layer in order to achieve action in the underlying cell-layers.

During topical drug development it is crucial to evaluate this parameter and it is well known that the vehicle used in the formulation of the drug has a significant impact on the drug's ability to penetrate the stratum corneum. The models for stratum corneum penetration used in the development process should be predictive for human skin if the aim is to develop products for humans. Unfortunately, the stratum corneum of an experimental animal is typically very different to that of a human and this difference is due to the absence of human fur which has lead to the development of a thick human stratum corneum. For example, the mouse stratum corneum consists of three layers of dead keratinocytes while the human counterpart consists of fifteen layers.

Release Tests in Formulation Development

The Institute for Applied Dermato-Pharmacie at Martin-Luther Universitat in Halle (Saale), Germany (IAPD) was contacted and it was decided to test their in vitro release model as a tool for formulation development. The release model was developed by Professor Reinhardt Neubert (Neubert, R., Bendas, C., Wohlrab, W., Gienau, B., Furst, W. A multilayer membrane system for modelling drug penetration into skin Int J. Pharm. 75 (1991) 89-94; Knorst, M., Neubert, R., Wohirab, W. Release of urea from semisolid formulations using a multilayer membrane system. Drug Dev. Ind. Pharm. 23 (1997) 253-257) and has been used to evaluate the release of urea and dithranol and other dermatological drugs from different topical formulations. The model is based on the release of the active ingredient from the vehicle into a layer of gel-membranes of dolichol/propylene glycol manufactured to mimic human stratum corneum. The composition of the membranes was developed to give the same release profile of reference compounds as in explanted human skin. The in-vitro release method has been validated versus release methods in explanted human skin. (Neubert et al. and Knorst et al. above).

The release method is standardized and data obtained on a particular formulation can be used to predict if the formulation will have clinical efficacy (that is, if the release rate of the drug will be fast or slow). A fast release rate of a drug-substance from the formulation to the membranes predicts for fast release into stratum corneum and thus clinical efficacy of the drug formulation. The method has significant advantages over traditional release systems (such as Franz-cells) and was developed to compare generic formulations of drugs. The method has not yet obtained a regulatory status (i.e. a release profile of a drug from a generic formulation is not accepted in a registration file).

FIG. 1 provides a description of the multilayer membrane system (MMS)-method for evaluation of release of TriAc from the formulation into the membranes (see Neubert et al. and Knorst et al. above). In each of the experiments reported herein, around 10 mg of the formulation was placed on top of a pack of membranes. Samples were then incubated at 320 C for 30, 100 and 300 minutes (n=5). TriAc was then extracted from the membranes by shaking each membrane in absolute ethanol. The membrane was then removed and the ethanol-fraction was injected into an HPLC-system. The total amount of TriAc (as % fraction of total TriAc added in the cream) in the membranes was plotted as “TriAc-released” versus incubation time. AUC (Area Under Curve) in the interval 0-100 minutes was calculated. The plot to the right of the MMS-model description in FIG. 1 is a graphical representation of the release results obtained with Essex-cream and shows the area of interest for calculating AUC.

Table 2 shows the release data for a set of batches of F1 and two other TriAc formulations. TriAc was incorporated in Essex-cream dissolved in propylene glycol. TriAcana™ is a commercial formulation of TriAc (registered in France for obesity treatment). F1 was first produced as a pilot-batch in the formulation development program. Later, large scale batches of F1 containing 0.1% TriAc (F1A, F1B, F1C) or 0.03% TriAc (F1D, F1E, F1F) were manufactured on three occasions in accordance with GMP. F1TestA is a test batch where TriAc was added to the oil phase instead (oil phase addition of TriAc) and therefore this variant did not include isopropanol. In the test batch F1TestB TriAc was added to the oil-phase as well but the same amount of isopropanol as used in all other batches was also added to the cream.

Results and Conclusions

Based upon empirical observations by experts on the method (Professor R. Neubert, IAPD), a fast release predicts greater clinical efficacy. The fastest theoretical release that could be obtained in the MMS-model would be if 100% of the test compound were retrieved in the membranes after 30 minutes. However, such a fast release rate has never been seen in any studies with the MMS-model and a release of 40-50% of the active compound after 30 minutes (as with F1) is considered as superior to the values seen for the Essex-cream and for TriAcana™. An alternative way to compare release-rate from different products is to calculate the AUC (Area Under Curve) as % released×minute for the first 100 minutes. The AUC 0→100 min is around three times larger for F1 than for the other products Essex-cream and TriAcana™.

If the fastest theoretical release was obtained (100% in 30 minutes), the value of AUC after 100 min. would be 8500 (%×min.). Final batches of TriAc (0.03-0.10%) in F1 (i.e. F1A to F1F) all show a release rate larger than 50% of 8500. This is in contrast to less than 20% of 8500 for Essex-cream and TriAcana formulations.

The results obtained are very similar for different batches of F1 and this demonstrates the usefulness of the MMS-method as a tool for quality control to compare batch-to batch variations or to evaluate whether generic formulations of TriAc can be predicted to have the same clinical efficacy as F1.

The results obtained also indicate that the same percentage of TriAc is released from the low dosage form (0.03%) as from the high dosage form (0.1%). In addition, the results indicate that the release rate of TriAc from F1 is not dependent on how TriAc is added to the cream-base. The manufacturing process for F1TestB and F1TestA differed from the process for the other batches in that TriAc was added to the oil-phase during manufacture (see example 1) rather than being added as the last ingredient to the cooled cream-base. Moreover, the similarity in release rates between F1TestA and all other batches of F1 indicates that isopropanol may be omitted from the cream-base.

### Example 3

### Formulation Stability Studies

The shelf life of F1-TriAc was evaluated by measurement of the content of key ingredients and pH after storage up to 24 months (see Table 3). The particular example shown in Table 3 describes the low dosage formulation (0.03% TriAc) stored at 4° C. Similar results were obtained with the high dosage formulation (0.10% TriAc). The stability of creams produced on different occasions was also similar.

From these results it can be concluded that F1 is a suitably stable cream formulation for TriAc.

TABLE 3
Storage stability of F1-TriAc

| Months in | Concentration of Ingredients (% w/w) | | | | |
|---|---|---|---|---|---|
| Storage | TriAc | Methylparabens | Propylparabens | Imidurea | pH |
| 0 | 0.032 | 0.210 | 0.100 | 0.280 | 6.86 |
| 3 | 0.030 | 0.190 | 0.990 | 0.272 | 6.81 |
| 6 | 0.030 | 0.197 | 0.100 | 0.318 | 6.80 |
| 9 | 0.029 | 0.209 | 0.095 | 0.285 | 6.79 |
| 12 | 0.031 | 0.201 | 0.102 | 0.259 | 6.79 |
| 24 | 0.029 | 0.183 | 0.098 | 0.216 | 6.76 |

### Example 4

### Safety Studies

A local tolerance study using repeated epicutaneous administration for 4 weeks twice daily onto intact and abraded skin of Himalayan rabbits was performed. Three strengths of TriAc in F1 were tested: 0.03%, 0.1% and 0.3%. The control group was treated with the cream-base of F1. The dose was administered by epicutaneous application twice a day at a 6-hour interval. Cream (0.5 ml) was applied at each application site on every dosing occasion. In total, 24 rabbits were treated (3 males and 3 females in each dose group). The cream was applied to intact skin (left side) and to abraded skin (right side) in each animal. No substance-related local tolerance reactions were observed clinically in the rabbits during daily observations and necropsy. No mortality occurred. No substance-related influence was observed for behaviour and external appearance. Body weight of the rabbits was not influenced by the 4-week treatment with TriAc in F1. Food intake was within the normal range. No substance-related pathological changes were observed either macroscopically or microscopically. Thus, neither TriAc in F1 nor the cream base of F1 alone have any local irritating properties on the skin of rabbits.

### Example 5

### Efficacy Studies in Clinical Trials

A human clinical trial with TriAc in F1 has been completed. The trial was a single centre, phase I study of two doses of TriAc (0.03% or 0.1% w/w) in comparison with placebo (F1 cream base) on the effect on skin pro-collagen production. The trial was performed at the Department of Dermatology, Sahlgrenska University Hospital, Gothenburg, Sweden. The trial was a double blind, parallel group, comparative, randomized, single centre study. The volunteers were randomized to receive either 0.03% TriAc, 0.1% TriAc or placebo cream. There were six volunteers per treatment group. The abdominal area of the body was treated. The primary objective was to compare the change in skin pro-collagen types I and III.

It is known that topical betamethasone (a potent corticosteroid frequently used to treat various inflammatory dermatological conditions) leads to reduced synthesis of collagen in dermal fibroblasts. It has been demonstrated that three days of topical treatment with betamethasone (and with other potent corticosteroids) leads to a significant reduction (around 70% decrease from base-line) in expression of pro-collagen I (pro-collagens are precursors to collagen) and that the recovery is slow. Even after a 14 day corticosteroid-free period, pro-collagen production was decreased by 50% (Haapasaari K-M, Risteli J, Koivukangas V, Oikarinen A., Comparison of the effect of hydrocortisone, hydrocortisone-17-butyrate and betamethasone on collagen synthesis in human skin in vivo. Acta Derm Venerol (Stockholm) 75 (1995) 269-271). The precursor to another collagen (collagen III) is also known to be regulated by topical treatment with bethamethasone in a similar manner to pro-collagen I.

The amounts of pro-collagens (the aminoterminal propeptides of type I and type m collagens, PINP and PIIINP) in the dermis can be measured by radioimmunoassays on suction blister fluids (Kiistla U. Suction blister device for separation of viable epidermis from dermis. J Invest Dermatol 50 (1968) 220-5). The suction blisters were induced and the fluid in the blisters was collected for analysis.

FIG. 2 shows a representative response to treatment with F1-placebo or F1-TriAc (0.03%). The subjects' abdominal skin was treated with topical betamethasone (twice/day) for three days (day 0-3). The areas of skin were then treated with F1-placebo or with F1-TriAc (0.03%) respectively for 14 days.

Suction blister fluids were obtained on days 3, 10 and 17 and the content of PINP was determined and compared with baseline value. PINP content is shown in FIG. 2 as % of baseline value.

The results demonstrate that treatment with F1-TriAc (0.03%) restores PINP-expression in betamethasone treated skin faster than treatment with F1-placebo. Since PINP is a precursor to collagen this implies that treatment with F1-TriAc (0.03%) will increase the thickness and elasticity of the dermis and thus restore dermal atrophy.

1. A composition comprising at least one thyroid hormone compound or thyroid hormone-like compound, a hydrophilic phase-forming component, an ethanolamine and at least two emulsifying or emollient excipients selected from the group consisting of mineral oil, C₁₂-C₂₄ alcohols, C₁₂-C₂₄ carboxylic acids, C₁-C₈ branched or linear alkyl esters of C₁₂-C₂₄ carboxylic acids, glyceryl esters of C₁₂-C₂₄ carboxylic acids, macrogol ethers, polyethylene glycol esters of C₁₂-C₂₄ carboxylic acids, sorbitan esters of C₁₂-C₂₄ carboxylic acids (Span compounds) and polyoxyethylenated sorbitan esters of C₁₂-C₂₄ carboxylic acids (Tween compounds).

2. A composition according to claim 1 in which the ethanolamine is triethanolamine.

3. A composition according to claim 1 in which the hydrophilic phase-forming component comprises water.

4. A composition according to claim 1 in which the emulsifying or emollient excipients are selected from octyl palmitate, isopropyl myristate, isopropyl palmitate, cetostearyl alcohol, Cetearth-20, Cetomacrogol 1000, glyceryl monostearate, cetyl alcohol, stearic acid, glyceryl stearate and PEG-100 stearate.

5. A composition according to claim 1 in which the ethanolamine is present in the composition at a level of 0.1 to 5% w/w.

6. A composition according to claim 1 which further comprises a humectant.

7. A composition according to claim 6 in which the humectant is present at a concentration of 1 to 5% w/w.

8. A composition according to claim 1 which further comprises a thickening agent.

9. A composition according to claim 8 in which the thickening agent comprises a carbomer or carbopol.

10. A composition according to claim 8 in which the thickening agent is present at a concentration of 0.1 to 1% w/w.

11. A composition according to claim 1 which further comprises one or more preservative ingredients.

12. A composition according to claim 11 in which the preservative ingredients are selected from methylparaben, propylparaben, imidurea and 1-(3-chloroallyl)-3,5,7-triaza-1-azoniaadomaniane chloride.

13. A composition according to claim 1 which further comprises EDTA.

14. A composition according to claim 1 which further comprises one or more antioxidants.

15. A composition according to claim 14 in which the antioxidants are selected from butylated hydroxyanisole, butylated hydroxytoluene, n-propyl trihydroxybenzoate, t-butylhydroquinone and dl-α, δ, γ-tocopherol.

16. A composition according to claim 1 which further comprises a UV-filter component.

17. A composition according to claim 16 in which the UV-filter component comprises one or more compounds selected from aminobenzoic acid, dioxybenzone, oxybenzone, sulisobenzone, diethanolamine methoxycinnamate, ethyldihydroxypropyl aminobenzoate, glyceryl aminobenzoate, octyl dimethyl aminobenzoate, trolamine salicylate and octyl methoxycinnamate.

18. A composition according to claim 1, wherein said composition has a pH of 6.1 to 7.1.

19. A composition according to claim 18 having a pH of 6.8 to 7.1.

20. A composition according to claim 1 in which the at least one thyroid hormone compound or thyroid hormone-like compound is selected from Tri-iodothyronine (3,5,3′-triiodothyronine) (T3); D and L tetraiodothyronine-thyroxine (T4); 3,3′,5′ tri-iodothyronine (reverse T3); 3,3′-diiodothyronine; T3 T4 analogues such as 3, 5,3′-Triiodothyroacetic acid; 3, 5, 3′-Tetraiodothyroacetic acid; 3, 5, 3′-Triiodo-L-thyronine; 3, 5, 3′-Triiodo-L-thyronine methyl ester; 3, 5, 3′-Triiodo-L-thyronine hydrochloride; L-thyroxine; L-thyroxine hydrochloride; L-T3; T4Fo; Tetrac (3-[4-(4-hydroxy-3,5-diiodophenoxy)-3,5-diiodophenyl]acetic acid); Triac ([4-(4-hydroxy-3-iodophenoxy)-3,5-diiodophenyl]acetic acid); Tetraprop; Triprop ([4-(4-hydroxy-3-iodophenoxy)-3,5-diiodophenyl]propionic acid); T4Bu; T3Bu; Thyroxamine; Triiodothyronamine; L-3‘-Tl’; L-3′-T1; L-3,5′-T2; L-3,3′-T2; L-3,3′,5′-T3; DL-Br2I; L-Br2iPr; L-Me2I; L-Me3; L-Me4; L-Me21Pr; DL-IMEI; L-3,5-Dimethyl-3′-isopropylthyronine (DIMIT); DL-BPT4; B-triac; BP-tetrac; DL-SBT3; DL-SBT4, DL-MBT3, MB-tetrac; T2; T2F; T2CI; T2Br; T3; T2Me; T2Et; T21Pr; T2nPr; T2sBu; T2tBu; T2IBu; T2Phe; T20H; T2NO2; T2F2; T2CI2; T4; T2Me2; 3, 5, 3′,5′-tetraiodo-D-thyronine; 3,5,3′-Triiodo-D-thyronine; 3,5-Diiodo-4-hydroxyphenylpropionic acid (DIHPA); Aryloxamic acids; (arylamino) acetic acids; arylpropionic acids; arylthioacetic acids; (aryloxy) acetic acid; 3,3′-T2; 3,5-T2; 3′-5′-T2; (5-Benzyloxy-2-methoxyphenyl)-(2-methoxypyrimidin-5-yl)-methanol; Benzyloxy-2-methoxyphenyl)-(6-methylpyridin-3-yl)methanol; (5-Benzyloxy-2-methoxyphenyl)-(5-bromo-2-methoxypyridin-4-yl)methanol; (5-benzyloxy-2-methoxyphenyl)-2,6-difluoropyridin-3-yl)methanol; (5-Benzyloxy-2-methoxyphenyl)-(2-methoxypyridin-4-yl) methanol; 4-Methoxy-3-[(2-methoxypyrimidin-5 -yl)methyl]phenol; 4-Methoxy-3-[(6-methylpyrid-3-yl)methyl]phenol; 5-Benzyloxy-2-methoxybenzyl Bromide; (5-Benzyloxy-2-methozyphenyl)-(6-chloropyridazin-3-yl)-acetonitrile; 4-Benzyloxy-2-[2-methoxythiazol-5-yl)methyl]anisole; 6-[(5-Hydroxy-2-methoxyphenyl)methyl]thiazol-2-(3H); 3′-Heteroarylmethyl-4′-)-methyl-3,5-dinitro-N-trifluoro-acetyl-L-thyronine Ethyl Esters; 3′-heteroarylmethyl-3,5-di-iodo-4′)-methyl-N-trifluoro-acetyl-L-thyronine Ethyl Esters; 3′-heteroarylmethyl analogues of 3,3′,5-tri-iodo-L-thyronine (T3); 3′-substituted derivatives of the thyroid hormone 3,3′,5-triiodo-L-thyronine (T3); α-methyl-3,5,3′-triiodothyroacetic acid, α-methyl-3,5,3′-triiodothyropropionic acid, and α-methyl-3,5,3′5′-tetraiodothyropropionic acid; methylene- and carbonyl-bridged analogs of iodinated thyronines or thyroacetic acids; or iodinated benzofurans; 3,5-diiodo-4-(2-N,N-diethylaminoethoxy)phenyl-(2-butylbenzofur-3-yl)methanol hydrochloride; 2-methyl-3-(3,5-diiodo-4-(2-N,N-diethylaminoethoxy)-benzoyl) benzofuran hydrochloride; 2-n-butyl-3-(3,5-diiodo-4-carboxymethoxy-benzoyl)benzofuran; 2-methyl-3-(3,5-diiodo-4-carboxymethoxy-benzyl)benzofuran; [4′-hydroxy-3′-iodo-3, 5 diido-4-(2-N,N-dimethylamino-(ethoxy)benzophenon hydrochloride; 2-butyl-3-(3-iodo-4-hydroxybenzoyl)benzofuran; 4′4-dihydroxy 3′3,5-triiodo-diphenylmethane; 3,5-diiodo-4-(2-N,N-diethylaminoethoxy)phenyl-; -(2-butylbenzofur-3-yl)methanol hydrochloride; 2-n-butyl-3-(3,5-diiodo-4-carboxymethoxy-benzoyl)benzofuran; 2-methyl-3-(3,5-diiodo-4-hydroxy-benzoyl)benzofuran; 2-methyl-3-(3,5-diiodo-4-carboxymethoxy-benzyl)benzofuran; 4′hydroxy-3′-iodo-3,5 diiodo-4-(2-N,N-dimethylamino-ethoxy)benzophenon hydrochloride; 2-butyl-3-(3 -iodo-4-hydroxybenzoyl)benzofuran; 4′,4-dihydroxy-3′3,5-triiodo-diphenylmethan; 3,5-diethyl, 3′-isopropyl thyronine (DIET); and IpTA2 (3,5 diiodo-3′ isopropyl thyroacetic acid) and pharmacologically acceptable salts and derivatives thereof.

21. A composition according to claim 20 in which one of the thyroid hormone or thyroid hormone-like compounds is triodothyroacetic acid (TriAc).

22. A composition according to claim 21 in which the TriAc is present at a concentration of 0.001 to 0.3% w/w.

23. A composition according to claim 22 in which the TriAc is present at a concentration of 0.01 to 0.3% w/w.

24. A composition according to claim 1 comprising (% w/w) water (77 to 79), octyl palmitate (5.0), cetostearyl alcohol (1.0), glyceryl monostearate (6.0), cetyl alcohol (2.0), stearic acid (3.0), sorbitol (2.8), triethanolamine (0.4 to 0.8), methylparaben (0.2), propylparaben (0.1), imidurea (0.3), disodium EDTA (0.2), isopropanol (0.7), carbopol 980 (0.2) and TriAc (0.03 to 0.1).

25. A composition according to claim 1 which, when placed in contact with dolichol/propylene glycol gel membranes according to the MMS-method, exhibits a release rate of the thyroid hormone or thyroid hormone-like compound which exceeds 25% of the theoretical maximum release rate for that method calculated as AUC from 0 to 100 minutes.

26. A method of predicting the efficacy of topical compositions according claim 1, the method comprising contacting the compositions with a stack of dolichol/propylene glycol gel membranes, incubating the compositions and membranes to allow diffusion of the thyroid hormone or thyroid hormone-like compounds into the membranes, extracting the diffused thyroid hormone or thyroid hormone-like compounds from the membranes and measuring the amount of extracted diffused thyroid hormone or thyroid hormone-like compounds.

27. A method of predicting the efficacy of topical compositions comprising thyroid hormone or thyroid hormone-like compounds, the method comprising contacting the compositions with a stack of dolichol/propylene glycol gel membranes, incubating the compositions and membranes to allow diffusion of the thyroid hormone or thyroid hormone-like compounds into the membranes, extracting the diffused thyroid hormone or thyroid hormone-like compounds from the membranes and measuring the amount of extracted diffused thyroid hormone or thyroid hormone-like compounds.
